(19) United States
(12) Patent Application Publication
(10) Pub. No.: US 2004/0091604 A1
(43) Pub. Date: May 13, 2004

(54) Bioactive surface for titanium implants

(76) Inventors: Donald J. Dempsey, Newbury, MA (US); William C. Quist, Brookline, MA (US); Lori M. Anderson, Toronto (CA); Matthew D. Phaneuf, Ashland, MA (US); Frank W. LoGerfo, Cambridge, MA (US)

Correspondence Address: David Prashker, Esq., DAVID PRASHKER, P.C., P.O. Box 5387, Magnolia, MA, 01930, US

(21) Appl. No.: 10/438,542

(22) Filed: May 15, 2003

Related U.S. Application Data
(63) Continuation of application No. PCT/US02/22734, filed on Jul. 17, 2002.
(60) Provisional application No. 60/306,976, filed on Jul. 19, 2001.

(51) Int. Cl. A61L 2/00

(52) U.S. Cl. 427/2.27; 623/1.46

(57) ABSTRACT

The present invention is a broadly applicable methodology for making a bioactive titanium surface which would be clinically-acceptable and effective as either an anti-thrombin, thrombolytic or growth promoting surface coating, or any combination of these. The bioactive surface can be prepared using any material comprising titanium in whole or in part; is suitable for inclusion upon the exposed surfaces of surgically implantable prostheses comprising titanium; offers a means for avoiding systemic anticoagulation therapy to reduce thrombus formation and thromboembolism in the living subject receiving a surgically implanted prosthesis; and provides a means to induce cellular attachment and proliferation onto the titanium surface of the implant.

### PRIORITY FILING

The present invention was first disclosed in an application filed Jul. 19, 2001 as U.S. Provisional Patent No. 60/306,976.

### RESEARCH SUPPORT

The research for the present invention was supported by a National Institutes of Health grant (1R43HL6307-01A1). The government has certain rights in the invention.

### FIELD OF THE INVENTION

The present invention is concerned generally with improvements of biocompatible and surgically implantable prostheses; and is directed to the generation of active biosurfaces which present substantial biologic properties such as anti-thrombin, thrombolytic or growth promoting properties for prosthetic articles and devices comprised in whole or in part of titanium.

### BACKGROUND OF THE INVENTION

Titanium (Ti) is the primary metal comprising such implantable devices such as mechanical heart valves, artificial organs (i.e. total implantable heart, left ventricular assist devices) access ports and surgical clips. Ti has advantageous bulk and surface properties: a low modulus of elasticity (needed for rigid applications), a high strength to weight ratio (versus stainless steel), excellent resistance to corrosive environments and forms stable oxides immediately upon exposure to oxygen. This corrosion resistance is due to an oxide layer found on all Ti surfaces. Although Ti is a highly reactive metal, it forms stable oxides immediately upon exposure to ambient conditions. This biocompatible film is the interface present at the cellular level [Brown SA, Lemons JE. Medical Applications of Titanium and its Alloys: The Material and Biological Issues. American Society for Testing Materials, Philadelphia, Pa., 1996].

Even with these positive attributes, Ti implants are prone to surface thrombus formation. For example, cardiac valve replacement and implantation of an artificial organ as a bridge to transplant are increasing in frequency due to an aging population. For mechanical heart valves, greater than 50,000 valves per year are projected for implantation over the next ten years. Associated with these devices is the risk of failure due to primary thrombosis and distant thromboembolic complications. The cause of these complications is due to lack of spontaneous endothelial healing of the device, altered hemodynamics in both systole and diastole resulting in zones of stasis and the inherent thrombogenicity of the biomaterial itself used in the device. Further, the material incorporated into the valve-sewing ring adds yet another source for thrombus formation. The anatomic site of replacement also affects thrombosis risk, with the risk being greater for mitral replacement over aortic replacement [Chesebro JH, Fuster V. Valvular heart disease and prosthetic heart valves. In Fuster V, Verstraete M (Eds.) Thrombosis in Cardiovascular Disorders, Philadelphia, W B Saunders 1992, 198]. Thrombotic complications are seen with all types of mechanical valves and are independent of valve design and composition. Schoen et. al. have attributed up to 20% of mechanical valve failures due to thrombus related events [Schoen FJ. Surgical Pathology of removed natural and prosthetic heart valves. Hum Pathol 18:558, 1987]. As a result, patients undergoing mechanical cardiac valve replacement must be anticoagulated due to the risk of thrombosis and thromboembolism [Edmunds LH. Thrombotic and bleeding complications of prosthetic heart valves. Ann Thoracic Surg 1987;44:430, Chesebro JH, Fuster V. Thromboembolism in heart valve replacement. In Kwaan HC, Bowie EJW (Eds.) Thrombosis Philadelphia, W B Saunders, 1982, 146, Phillips SJ. Thrombogenic influence of biomaterials in patients with the Omni series heart valve: pyrolytic carbon versus titanium. ASAIO J 2001;47(5):429]. These patients run the risk of bleeding complications associated with anticoagulation [Chesebro JH, Fuster V. Valvular heart disease and prosthetic heart valves. In Fuster V, Verstraete M (Eds.) Thrombosis in Cardiovascular Disorders, Philadelphia, W B Saunders 1992, 198, Levine MN, Raskob G, Hircsh J. Hemorrhagic complications of long-term anticoagulant therapy. Chest 1988; 95(2): 26S]. Even with anticoagulation, there remains a significant risk for thromboembolic complications in the range of 1-2%. Platelet and thrombin deposition on the mechanical valve surface dominates the initial surface interaction. Modification of material design to alter this interaction has, to date, been largely unsuccessful.

Implantation of access ports has become increasingly employed for patients that require hemodialysis, long-term drug delivery or phlebotomy. These ports are flushed with a heparin-antibiotic solution (heparin lock) in order to prevent venous thrombus formation/infection within the port. However, even with this procedure, reported thrombosis rates range from 1.5% to 12.5% [Biffi R, de Braud F, Orsi F, Pozzi S, Mauri S, Goldhirsch A, Nole F, Andreoni B. Totally implantable central venous access ports for long-term chemotherapy. A prospective study analyzing complications and costs of 333 devices with a minimum follow-up of 180 days. Ann Oncol 1998;9(7):767, Burbridge B, Krieger E, Stoneham G. Arm placement of the Cook titanium Petite Vital-Port: results of radiologic placement in 125 patients with cancer. Can Assoc Radiol J 2000;51(3):163, Biffi R, de Braud F, Orsi F, Pozzi S, Arnaldi P, Goldhirsch A, Rotmensz N, Robertson C, Bellomi M, Andreoni B. A randomized, prospective trial of central venous access ports connected to a standard open-ended or Groshong catheters in adult oncology patients. Cancer 2001;92(5):1204]. This complication results in replacement of the access device.

Efforts to combat thrombus formation involve coating the Ti implants with pyrolytic carbon, non-specific binding of proteins to Ti surfaces and altering the bulk surface properties of metal. The most recent research involving Ti materials centered on non-specifically binding factors to the Ti surface and monitoring subsequent effects of the release [Linneweber J, Kawamura M, Motomura T, Ishitoya H, Nonaka K, Ichikawa S, Hellums JD, Nose Y. Effect of albumin-bound GPIIb/IIIa inhibitor on shear-induced platelet deposition on titanium. ASAIO J 2001;47(2):171, Kawamura M, Linneweber J, Ishitoya H, Motomura T, Mikami M, Shinohara T, Kawahito S, Nonaka K, Hellums JD, Nose Y. Pharmacological approach to prevent high shear stress induced thrombus formation on titanium surface. ASAIO J 2001;47(2):171]. Thus, none of recent published research has resulted in any direct immobilization of a biologically-active agent to Ti in order to localize the effects of the agent to the immediate surface.

Clearly therefore, there has been and today remains a long-standing recognition and need for new prosthetic mechanical devices having improved anti-thrombin attributes, anti-thromboembolic or cell growth promoting capabilities. Were such new mechanical prostheses to be developed, their anti-thrombin attributes would facilitate mechanical valve replacement surgery; and would avoid the present use of acute systemic anticoagulation; and would reduce the occurrence and severity of bleeding complications for the patient.

### SUMMARY OF THE INVENTION

The present invention has multiple aspects and alternative definitions.

A first aspect of the invention provides a method of making a bioactive surface for a material comprised of titanium, said method comprising the steps of:

obtaining access to at least one exposed surface of a material comprised of titanium;

oxidizing said exposed surface of the material comprised of titanium with at least one oxidizing agent to yield a titanium oxide surface layer;

combining said titanium oxide surface layer with at least one organosilane coupling agent to produce a plurality of organic reactive sites disposed at the surface of the material;

reacting said organic reactive sites disposed at the surface of the material with at least one composition having not less than one pendant amino group as part of its formulation and structure to yield a plurality of pendant amino groups immobilized at the material surface which are functionally available for subsequent chemical reaction;

binding at least one biologically active agent to said immobilized pendant amino groups to generate a bioactive surface for the material.

A second and alternative aspect of the invention provides a method of making a bioactive surface for a prosthetic implant comprised of titanium, said method comprising the steps of:

obtaining access to at least one exposed surface of a prosthetic implant comprised of titanium;

oxidizing said exposed surface of the prosthetic implant comprised of titanium with at least one oxidizing agent to yield a titanium oxide surface layer;

combining said titanium oxide surface layer with at least one organosilane coupling agent to produce a plurality of organic reactive sites disposed at the surface;

reacting said organic reactive sites disposed at the surface with at least one composition having not less than one pendant amino group as part of its formulation and structure to yield a plurality of pendant amino groups immobilized at the surface which are functionally available for subsequent chemical reaction;

joining at least one bifunctional linking molecule to said pendant amino groups immobilized at the surface; and

binding at least one biologically active protein to said joined bifunctional linking molecule to generate a bioactive surface for the prosthetic implant.

### BRIEF DESCRIPTION OF THE FIGURES

The present invention may be easily understood and better appreciated when taken in conjunction with the accompanying drawing, in which:

FIG. 1 is a graph illustrating the qualitative and quantitative determination of amino groups on a prepared Ti-Ep-PEI surface;

FIG. 2 is a graph illustrating the amine content of prepared Ti-Ep-PEI surfaces;

FIG. 3 is a graph illustrating the degree of rHir binding to prepared Ti-Ep-PEI surfaces under different reaction conditions;

FIG. 4 is a graph illustrating the differences in active anti-thrombin activity of non-specifically bound and covalently bound rHir at the biosurface; and

FIG. 5 is a graph illustrating the anti-thrombin activity of the active biosurface to different concentrations of thrombin in-vitro.

FIG. 6 is a graph illustrating the degree of VEGF binding to prepared Ti-Ep-PEI surfaces under different reaction conditions

### DETAILED DESCRIPTION OF THE INVENTION

The present invention is a broadly applicable method for making a bioactive surface, such as an effective anti-thrombin coating, which is clinically-acceptable and is suitable as the exterior surface(s) of a surgically implantable prosthetic article or mechanical device.

The capability to create a bioactive surface, exemplified herein by an anti-thrombin biosurface, is generated by and results from the present technique and procedures; and is intended for all titanium-containing materials, alloys, or prosthetic implants without regard to their dimensions, design structure, or function(s). For instance, after an anti-thrombin biosurface has been prepared, the material or prosthetic implant will provide clinically-effective anti-thrombin properties and anti-thromboembolism attributes; as well as allow a reduction in currently used levels of systemic anticoagulation agents, thereby markedly diminishing both the severity and duration of bleeding complications for the patient.

I. Component Compositions Employed In Practicing The Methodology

A variety of different compositions, compounds and molecules are employed as work pieces and reactive chemical components in the present method to make a biologically active coating layer and biosurface for a sheet, a prosthetic article, or a mechanical device comprising titanium. Each of these substances employed as intermediate reactants will be disclosed and described in detail as to its formulation, its reactive properties, and its relationship in the formation and manufacture of the bioactive surface as a whole.

A. Titanium Metal And Titanium Alloys

Titanium (Ti) is a metal and an alloy constituent which has been used in many biomedical applications such as left ventricular assist devices, heart valves, dental implants and bone replacements. It has advantageous bulk and surface properties: a low modulus of elasticity (needed for rigid applications), a high strength to weight ratio (versus stainless steel) and excellent resistance to corrosive environments. This corrosion resistance is due to an oxide layer found on all Ti surfaces.

Although Ti is a highly reactive metal, it forms stable oxides immediately upon exposure to ambient conditions. This biocompatible film is the interface present at the cellular level [Brown, S. A. and J. E. Lemons, Medical Applications of Titanium and its Alloys: The Material and Biological Issues, American Society for Testing Materials, Phila. Pa, 1996].

Currently, bonding to Ti devices is mainly a physical attachment which is created by cellular ingrowth into a convoluted metal surface [Doherty et al., Biomaterials-Tissue Interfaces, Elsevier, Amsterdam, 1992]. Since adhesion at the interface of metal and tissue is often the weak link in cellular binding, treatment of the metal surface is of vital importance in improving the strength, reliability, and environmental resistance of the interfacial bond. The hydrophilic nature of the Ti oxide layer potentiates water penetration at the interface, thus weakening the cellular/metal bond.

In the past, various methods for Ti surface modification prior to surface bonding have been attempted, including the use of various primers and/or pickling baths used to oxidize the surface. Common oxidative methods employed previously include exposure of the surface to peroxides and/or inorganic acids [Walivarra et al., Biomaterials 15:827 (1994)]. Among these is an ASTM protocol F68 that uses nitric acid for the passivation of Ti devices as implants. However, additional studies have demonstrated that Ti devices treated by this ASTM protocol F68 have been subject to trace metal uptake. Thus, this conventionally known protocol cannot be employed, due to these findings; and an entirely new approach must be undertaken as follows.

Oxidation of Titanium Surfaces:

Oxidation of freshly abraded Ti surfaces occurs in less than 10 minutes when exposed to water. The repassivated surface formed is a 3-6 nm layer composed of titanium oxide [Hernandez et al., Appl. Surf. Sci. 68:107 (1993)]. This passivated film on the surface consists of two layers. The inner layer consists of TiO₂ and the remainder is a mixture of titanium oxy-hydroxide or hydrates. Oxygen atoms in the hydroxyl group are located mainly in the outer part of the surface film while dehydration occurs inside the surface film forming TiO₂ [Hanawa et al., J. Biomed. Mater. Res. 40:530 (1998)].

The overall chemical result is stated by Reaction I below.

Reaction I: 2TiO(OH)₂→TiO₂+2H₂O (dehydration)

Thus, efforts to promote oxidation are better served by controlling the type of the oxidation layer as well as its purity. This is best accomplished: first, by removing the passivated layer formed under ambient conditions; and, second, by controlling the formation of the repassivated layer using defined reaction parameters and pure reagents, thereby reducing absorption of contaminants into the oxide layer normally present under ambient conditions.

Several experiments performed using 30% hydrogen peroxide as an oxidizing agent have resulted in a darkening of the Ti surface, with the appearance being altered significantly with increased immersion times (blue gray appearance). However, after silanization (as described hereinafter), one could not detect any appreciable acid red uptake, indicating that no amine groups were present on the titanium surface.

A Preferred Oxidation Technique:

An oxide free titanium surface can be obtained by using inorganic acids, since these acids dissolve TiO₂. A fresh oxide layer can then be returned by subsequent treatment with de-ionized water, followed by dehydration at elevated temperatures. Treatments using hydrochloric and/or sulfuric acids yielded similar blue gray surfaces, as exhibited by the peroxide treatment. In contrast to the hydrogen peroxide experiments, color changes by acid red dye uptake were readily apparent after silanization, indicating the presence of amine groups. Therefore, acid etching is the preferred oxidation method due to: 1) more uniform dye uptake by the silanized segments and 2) the storage of large quantities of acids is more easily accommodated than large quantities of hydrogen peroxide (30%).

B. Organosilane Coupling Compounds

Organosilane coupling compounds, R—Si—(OH)₃ where R is an organic reactive site, are utilized herein as an intermediate entity in the formation of bioactive surfaces having effective biologic properties; and such organosilane coupling compounds offer and provide the requisite reactive entities and reactions for this purpose in the present methodology.

Organosilane Reaction Chemistry:

Reaction of alkylsilanols with surface hydroxyl groups has been successfully employed previously in the science of chromatography. Chemical surface modification of silicon oxide surfaces by silanization is a well known technique and is typically used for the preparation of stable column beds for liquid chromatography [Grushka, E. and E. Kikta, Anal. Chem. 49:1004A (1977)].

In the commonly used silanization technique, Silane is reacted with the surfaces in a liquid phase. If strict anhydrous conditions do not prevail, however, this technique often results in polymerization of the Silane and instability of the Silane films. However, under anhydrous conditions, the bond formed during the silanization of silicon dioxide materials has been reported to be hydrolytically stable if the silanization temperature exceeds 150° C. This enables the formation of a covalent bond between the Silane and the oxide covered surface [Jonsson et al., Thin Solid Films 124:117 (1985)].

The analogy of water infiltration encountered in the glass-fiber reinforcement industry is also applicable here. It was found that long-term water immersion problems of adhesive failures could be solved through the use of organosilane adhesion promoters. These coupling agents would form a bridge of chemical bonds between the inorganic glass surface and the organic epoxy resin matrix, thus preventing the entry of liquid water into the interface with concomitant debonding of polymer to fiber. An example of a coupling agent for glass-reinforced epoxy resin system is aminopropyltriethoxysilane, whose formula is:

(CH₂CH₂—O)₃—Si—CH₂CH₂CH₂NH₂

The ethoxy groups hydrolyze to form silanols (Si—OH), which then can condense with silanols on the glass fiber surface (Siₛ—OH) to form siloxane linkages, as follows:

Reaction I: 3H₂O+(CH₂CH₂—O)₃—Si—CH₂CH₂CH₂NH₂→(OH)₃—Si—CH₂CH₂CH₂NH₂+Ethanol↑

Reaction II: Siₛ—OH+(OH)₃—Si—CH₂CH₂CH₂NH₂→Si—O—Si(OH)₂—CH₂CH₂CH₂NH₂+H₂↑

The remaining silanol groups polymerize through a condensation reaction forming a polysiloxane film leaving the pendant amine. These pendant amines, in turn, then react with epoxy resin to complete the chemical bond. The stability of the surface siloxane linkage is equal to that of the siloxane linkages found in glass.

Chemical Interactions of Organosilane and Titanium:

Tetra alkyl titanates [Ti(OR)₄] can undergo trans-esterification with alcohols, with these reactions (alkyl titanates) being known to be hydrolytically unstable. However, the trans-esterification of Ti(OR)₄ with trimethylsilanol (R₃SiOH) yields a stable compound in the form of (R₃SiO)₄Ti [Raoul, F. and P. Cowe, The Organic Chemistry Of Titanium, Washington Butterworths, Great Britain, 1965, p. 21]. This reveals that the Si—O—Ti bond is hydrolytically stable under these specific reaction conditions.

Additionally, the alkoxysilane bond to surface SiOH (reported to be stable) can be hydrolytically unstable if silanization step occurs in the presence of excess water and/or a low reaction temperature [Grushka E. and E. Kikta, Anal. Chem. 49: 1004A (1977)]. Therefore, these parameters must be monitored in order to successfully bind Silane to the Ti surface.

The Nature of the Surface Modification:

The aim of the surface modification for the present method is not to create a monomolecular layer attached to the Ti surface. Rather, a uniform crosslinked film covalently attached to the titanium oxide surface having as many bonds as possible is most desirable. Currently, it is not known how many surface bonds are necessary to hold the crosslinked film in place. However, a film of this nature should prevent water and ion (Ca⁺², Na⁺¹, PO₄⁻³) migration from the biological fluid towards the underneath oxide surface, thereby keeping the oxide interface stable.

At room or low temperature, silanol groups compete with one another as well as with the reactive pendant group that, in this case, is the primary amine. Investigators have shown that the amine terminal end of the Silane molecule can associate with hydroxyls on the surface to form a weak electrostatic bond [Vandenberg et al., J. Colloid. Interface Sci. 147:103 (1991)]. These hydrolytically unstable attachments are the main cause of film dislodging. Under anhydrous surface conditions and extremely high temperatures, the thermally-sensitive hydrogen bonds will not form. Also, the amine group is stochiometrically deficient (1:3) to the silanol groups. Therefore, the proposed thermal and stochiometric conditions promote preferential attachment to the Ti surface via silanol reaction.

Note also that the condensation of the first hydroxyl group of a trisilanol readily occurs, forming a tetrasilanol. Then the condensation of second and the third hydroxyl group attached to the same silicon atom become increasingly more difficult [Bizios et al., J. Cell Physiol. 128:485 (1986)]. This sequence prolongs solution shelf life and reduces the volatility of the silane helping it to remain on the titanium surface during curing.

The Intended Goal of the Surface Modification:

The objective and purpose of the present methodology is to covalently bind biologically active proteins to the Ti surface, thereby creating a bioactive interface. The approach to creating this bioactive interface utilizes a variety of coupling agents that have been successfully used for many years in the plastics composite industry—specifically, Organosilane coupling agents.

Studies have shown that TiCl₄ vapor is very reactive to silica gel [Hair, M. L. and W. Hertl, J. Phys. Chem. 77:2070 (1973)]. Here, it was shown that TiCl₄ reacted with Si_{S}OH groups (where Si_{S} is the surface silicon), apparently yielding the Si_{S}—O—Ti group. In as much as the Si_{S}—O—Ti linkage (based on surface silicon and attached Ti) is so stable, it can be expected that the Ti_{S}—O—Si bond consisting of surface Ti and attached silicon is also stable. Moreover, since there is always a layer of chemisorbed oxygen or oxide on a Ti surface, the same compounds and methods used to form Si_{S}—O—Si bonds on glass can also be used to form Ti_{S}—O—Si bonds on Ti surfaces. Thus, the coupling agents historically used to chemically link glass to various polymers can be used to bind Ti to the same polymers; or carried further, to bind proteins to Ti by using appropriate crosslinkers.

Organosilane Coupling Agents:

A wide range and variety of silane coupling agents are conventionally known and suitable for use in this method. A representative, but not-exhaustive, listing of useful organosilane coupling agents is given by Table 1 below.

In addition, the person ordinarily skilled in this art will recognize that considerable choice, variation and modification of the reagents employed as well as in exposure and reaction times, in concentrations of reactants and reaction conditions (of temperature and/or pressure), and in the other details described within the disclosed experiments, are both possible and sometimes even desirable. Accordingly, all such choices, alterations and modifications are deemed to be within the skill of the ordinary practitioner in this field, and are within the intended scope of the present invention.

TABLE 1
Organosilane Coupling Agents

Allyldimethyldichlorosilane, Allyltrichlorosilane, Allyltriethoxysilane,
Allyltrimethoxysilane, 4-Aminobutyldimethylmethoxysilane, 4-
Aminobutyltriethoxysilane, (Aminoethoxyaminomethyl)phenyl-
trimethoxysilane, N-(Aminoethyl)-3-
aminopropylmethyldimethoxysilane, N-(Aminoethyl)-3-
aminopropylmethyltrimethoxysilane, N-(6-Aminohexyl) aminopropyl-
trimethoxysilane, 3-Aminopropyldimethylethoxysilane, 3-
Aminopropylmethyldiethoxysilane, 3-aminopropyltriethoxysilane, 3-
Aminopropyltrimethoxysilane, 2(3,4-
Epoxycyclohexyl)ethyltrimethoxysilane, (3-
Glycidoxypropyl)bis(trimethylsiloxy)-methylsilane, 3-
Glycidoxypropyldiisopropylethoxysilane, 3-
Glycidoxylpropyldimethoxyethoxysilane,
(3-Glycidoxypropyl)methyldiethoxysilane, 3-
Glycidoxypropyltrimethoxysilane, 3-Glycidoxypropyltriethoxysilane,
(Mercaptomethyl)dimethylethoxysilane,
(Mercaptomethyl)methyldiethoxysilane, 3-
Mercaptopropylmethyldimethoxysilane, 3-
Mercaptopropyltrimethoxysilane, 3-Isocyantopropyltriethoxysilane.

C. Cross-Linking Compositions Having at Least One Pendant Amino Group Available For Subsequent Chemical Reaction

A second chemical intermediate employed herein as a cross-linking agent in the formation of an active biologic surface suitable for a prosthetic implant are those chemical compositions having at least one, and preferably multiple, pendant amino groups available for subsequent reaction. This class of chemical compound is to be added to and reacted with a previously formed titanium-siloxane linkage Ti_{S}—O—Si(OH)n)R then disposed upon the exterior surface(s) of a material, or the preformed prosthetic article or device. The result and intended consequence of combining these reactants is the cross-linking, and covalent binding, and permanent immobilization of this intermediate molecule to the exposed surface(s) of the structure; with a concomitant availability of the pendant (single or multiple) amino groups within the molecular formulation—the pendant amino group(s)—remaining chemically free and functional for subsequent reaction with other compositions.

The minimal and essential requirements of such intermediate cross-linking agent compounds are few and include:

(i) The capability to bind covalently with one or more organic reactive site then existing and disposed upon the exposed surface(s) of a titanium based material or prosthetic entity;

(ii) The existence of at least one, and preferably multiple, free and functional amino groups pendant within the formulation and structure of the molecule; and

(iii) The ability of the pendant single or multiple amino groups within the composition to react subsequently with and bind covalently to a chosen polypeptide or protein having recognized biological properties.

To demonstrate merely the diverse range and wide variety of these intermediate molecules and cross-linking agents, a representative, but non-exhaustive, listing is given by Table 2 below.

TABLE 2
Cross-linking compounds having at least one pendant amino group
available for subsequent reaction

PolyethyleniminesMn 400 to 10,000;
(Polypropyleneglycol) bis(2-Aminopropyl ether) Mn 200 to 4000;
Ethylenediamine;
1,3 Propylenediamine;
1,2 Propylenediamine;
Neopentadiamine;
Butylenediamine;
Pentylenediamine;
Hexamethylenediamine;
Octamethylenediamine;
Diethylenetriamine;
N-(2-Aminopropyl)-1,3-propanediamine;
N-(3-Aminopropyl)-1,3-propanediamine;
N,N″-1,2-Ethylene bis(1,3-propanediamine)Tetraethylenepentamine.

D. A Modifying Bifunctional Linking Molecule

A third chemical intermediate employed herein as a reactant is at least one modifying bifunctional linking molecule which is suitable: first, for covalent reaction and juncture with the pre-existing pendant amino group(s) then immobilized at the material surface; and second, for subsequently binding a biologically active protein of choice thereto. In the present method, this intermediate reaction and chemical event occurs via the covalent bonds and cross-linking junctures provided and formed by one or more bifunctional linking molecules.

The term “bifunctional linking molecule” is defined herein as a crosslinking composition or chemical agent having the ability to bind to two reactive groups or moieties found on either the same entity or on different entities. The bifunctional linking molecule will thus serve to connect these two reactive groups sterochemically; and, as an intermediate, join the two reactive groups together as a coupled and unified chemical structure. By definition, a heterobifunctional linking molecule or agent is one that binds to two different types of reactive groups and joins them together as a unified structure. Conversely, if the bifunctional linking molecule binds two similar or identical reactive groups, it is referred to as a homobifunctional linking molecule or agent.

A wide range and variety of heterobifunctional and homobifunctional linking molecules are conventionally known in the scientific literature and are commercially available. Thus, some representative heterobifunctional linking molecules or agents suitable for use in the instant methodology include, but are not limited to: sulfosuccinimidyl 4-(N-maleimidomethyl) cyclohexane-1-carboxylate (sulfo-SMCC); succinimidyl 4-(N-maleimidomethyl) cyclohexane-1-carboxylate (SMCC); N-succinimidyl-3-(2-pyridyldithio) propionate (SPDP); sulfosuccinimidyl 2-(7-azido-4-methylcoumarin-3-acetamide) ethyl-1,3′-dithiopropionate (SAED); 1-ethyl-3-(dimethylaminopropyl)-carbodimide HCl (EDC); and Traut's reagent (2-iminothiolane hydrochloride).

In addition, a diverse choice of homobifunctional linking molecules or agents can also be usefully employed in this methodology and include, but are not limited to: ABH; ANB-NOS; APDP; APG; ASIB; ASBA; BASED; BS³; BMH; BSOCOES; DFDNB; DMA; DMP; DMS; DPDPB; DSG; DSP; DSS; DST; DTBP; DTSSP; EDC; EGS; GMGS; HSAB; LC-SPDP; MBS; M₂C₂H; MPBM; NHS-ASA; PDPH; PNP-DTP; SADP; SAED; SAND; SANPAH; SASD; SDBP; SIAB; SMCC; SMBP; SMPT; SPDP; Sulfo-BSOCOES; Sulfo-DST; Sulfo-EGS; Sulfo-GMBS; Sulfo-HSAB; Sulfo-LC-SPDP; Sulfo-MBS; Sulfo-NHS-ASA; Sulfo-NHS-LC-ASA; Sulfo-SADP; Sulfo-SAMCA; Sulfo-SANPAH; Sulfo-SAPB; Sulfo-SIAB; Sulfo-SMCC; Sulfo-SMBP; and Sulfo-LC-SMPT.

The chosen bifunctional linking molecule or agent is first covalently reacted with and joined to the pre-existing pendant amino group(s) immobilized at the material surface, which are functionally available for chemical reaction; and second, reacted subsequently with at least one biologically active protein in a quantitative amount such that the desired degree of binding site density is achieved with the active protein of choice. The preferred binding site density is provided by that amount of bifunctional linking molecules (moles/gram) that optimizes the cross-linking reaction coverage of that surface for the subsequent covalent binding and juncture of the active protein of choice. The intended consequence and result of these bifunctional linking reactions is the covalent juncture and steroscopic immobilization of the chosen biologically active protein to the material surface; and the formation of a biologically activate surface for the material or prosthetic implant

E. A Recognized Form of Biologically Active Protein

A biologically active protein is the last and final reactant to be covalently linked to the activated titanium surface; and, after attachment, will demonstrably retain and possess its characteristic biological attributes and functions (such as thrombolytic activity or growth factor properties).

One such entity is any recognized form of Hirudin Protein. Preferably, a recombinant Hirudin (“rHir”) is employed, such as a 6.965 Da recombinant protein synthesized from the leech protein hirudin. The rHir is a most potent specific inhibitor of thrombin [Markwardt, F., Biochim Acta 44:1007 (1985)]: rHir has a demonstrable inhibitory action against the enzymatic, chemostatic, and mitogenic properties of thrombin [Fenton, J. W., Sem. Thromb. Hemost. 14:234 (1988); Fenton, J. W. and D. H. Bing, Sem. Thromb. Hemost. 12:200 (1986)]; and rHir has also been shown to have potent anti-thrombin activity after being covalently immobilized onto a Dacron surface [Phaneuf et al., Artif. Organs 22:657 (1998); Phaneuf et al., ASAIO J. 44:M653 (1998); Phaneuf et al., Biomaterials 18:755 (1997)] or to another biomolecule [Phaneuf et al., Thromb. Haemostas. 71:481 (1994); Phaneuf et al., Blood Coagulation And Fibrinolysis 5:641 (1994)].

This recognized form of leech anticoagulant holds several advantages over heparin: 1) rHir inhibits thrombin directly whereas heparin requires anti-thrombin III; 2) heparin enhances platelet aggregation; 3) rHir inhibits the uptake of thrombin into fibrin clots; and 4) heparin is regulated by platelet function. Thus, rHir is the preferred agent for covalent attachment in order to reduce or eliminate the thrombus formation on the surface of Ti implants. Heparin and agatroban are other anti-thrombin agents that can be used as well as other analogs/derivatives of these components.

The Underlying Rationale for using Anti-Thrombin Agents such as Hirudin:

The value and benefit of utilizing a recognized form of Hirudin protein lies in its ability to create a thrombo-resistant biomaterial—i.e., the capability to avoid and overcome the effects of thrombin enzyme activity.

Thrombin is a pivotal enzyme in the blood coagulation cascade; and constitutes the primary agent responsible for thrombus formation. The principal function of thrombin is the cleavage of fibrinogen to fibrin. Additionally, thrombin also functions as a smooth muscle cell mitogen; is chemotactic for monocytes and neutrophils; and is an aggregator of lymphocytes. This enzyme has also been shown to bind to endothelial cells, inducing the release of platelet-derived growth factor (PDGF)-like growth factors; and has been shown to be a potent platelet aggregator, stimulating the release of platelet factors. Thus, thrombin—beyond its role in clot formation—has tremendous secondary effects, which include the induction of inflammation at the site of synthesis and the enhancement of cellular proliferation or hyperplasia by various activation mechanisms, all of which are beneficial in wound healing but are extremely deleterious to biomaterial function.

Many attempts have been previously made to create a thromboresistant biomaterial surface by establishing a new biologic lining on the material surface that would “passivate” this acute reaction. A majority of surface modification studies to date have focused on covalent or ionic binding of the anticoagulant heparin either alone [Barcucci et al., Biomaterials 15:955 (1994)]; or in conjunction with other biologic compounds [Jacobs, H. and S. W. Kim, J. Pharm. Sci. 75:172 (1986)]; or with spacer moieties [Nojiri et al., ASAIO Transactions 36:M168 (1990)].

All of these previous attempts have had only limited and short-term success in creating a thromboresistant surface. Possible flaws with these types of surface modifications are: 1) thrombin is not directly inhibited therefore fibrinogen amounts remain constant on the material surface permitting platelet adhesion; 2) heparin-coated biomaterials may be subject to heparitinaeses potentially limiting long-term use; 3) non-specifically bound compounds are rapidly desorbed from the surface which is under high shear stress thereby exposing the thrombogenic biomaterial surface; 4) rapid release of these non-specifically bound compounds may create an undesired systemic effect; and 5) charge-based polymers may be “passivated” by other blood proteins such that the anticoagulant effects are acute. The use of heparin and other similar proteins, however, may be beneficially continued for short term uses or if the cost of Hirudin is prohibitive for the application.

For these reasons, any recognized form, type, or format of a Hirudin protein is deemed to be a highly effective and desirable thromboresistant material. Moreover, for purposes of practicing the present methodology, neither the true source, origin, or mode of procurement for the Hirudin protein is of importance; and neither the means of protein manufacture, nor the process by which the protein is prepared or made available in appropriate quantities has relevance or meaning for the present invention as a whole.

Application of Thrombolytic Agents to Titanium Surfaces:

Covalent linkage of thrombolytic or “clot-busting” agents to a titanium surface can also provide significant benefits for an implantable device. Such thrombolytic agents today include, but are not limited to, streptokinase and urokinase and prourokinase. These agents provide enzyme function by the activation of plasminogen to plasma in-situ; and such in-situ activation results in the cleavage of fibrin, whereby clot lysis occurs.

Thus, on exteriors such as the exposed surfaces of mechanical heart valves, stents and ventricular assist devices where clots are problematic, the immobilization of a thrombolytic agent results in cleavage of surface bound clots, thereby preventing thrombosis and/or thrombolytic events. This practice and protocol may also be used in conjunction with conventional anti-thrombin therapy in order to maintain a clot free surface for the implant in-vivo.

Application of Growth Factors to Titanium Surfaces:

The use of titanium metal and titanium alloys in bone replacements, dental implants, mesh for spinal fusion or surgical spikes, staples, nails have complications related to lack of cellular adhesion onto the surfaces. While convoluted surfaces permit cell migration toward the surface, direct tissue/surface interface is limited. Covalent linkage of one or more growth factors and/or adhesion molecules will therefore result in greater direct interaction of the cell wall with the surface of the implant.

Some useful growth factors include, but are not limited to the VEGF, FGF (basic or acidic), PDGF, ECGF, and BMP families. Suitable adhesion molecules include RGD peptides, ICAM, VCAM, PCAM, and other glycoproteins such as VEAI.

An exemplary demonstration of the juncture of VEGF to the titanium surface using the chemical reactant intermediates in series and the instant method is provided hereinafter. In addition, the use of such growth factors generally as described herein may be employed alone or in conjunction with the other proteins described previously above.

II. The Types of Materials And Prosthetic Implants Comprised Of Titanium

A wide range and variety of prosthetic articles, mechanical devices, surgical implants, and replacement parts are intended to utilize the present methodology for its anti-thrombin surface properties and advantages. An exemplary and representative, but non-exhaustive, listing is provided by Table 3 below.

TABLE 3
Exemplary Prosthetic Implants

Prosthetic Articles of Manufacture

valve housing chambers;
stents
ports for hemodialysis

Prosthetic Mechanical Devices

heart valves;
ventricular assist devices

Prosthetic Surgical Implements

dental implants;
surgical nails, spikes, and staples
mesh for spinal fusion

Prosthetic Replacement Parts

preformed bone replacements

### III. The Steps Comprising the Methodology as a Whole

The present invention is based on the premise that the layer of chemisorbed oxygen or oxide layer on a Ti surface can be utilized to form Ti_{S}—O—Si bonds on Ti implants using a coupling agent such as glycidyloxypropyltrimethoxysilane (Ep) to form Ti_{S}—O—Si-Ep bonds referred to as Ti-Ep. A hydrophilic compound containing multiple amine functional groups could then be covalently bonded to the prepared Ti-Ep surface. Subsequently, a potent bioactive agent such as anti-thrombin, thrombolytic or growth factor moiety can be covalently attached using specific crosslinkers. The preferred anti-thrombin agent is Hirudin protein, most desirably in the form of a recombinant Hirudin (rHir).

In order first, to demonstrate the validity of this process; and second, to make the most effective use of the procedures and chemical reactions available, a series of experiments were performed. The resulting empirical data observed and recorded thus provides the best mode of practicing the methodology known to date.

It will be expressly understood however, that the experiments and data presented hereinafter are merely representative and illustrative of the manipulative steps comprising the methodology as a whole; and are merely one example of the process for generating a bioactive surface for Ti materials and implants. In the experiments disclosed below, a thromboresistant surface is generated via covalent juncture of the potent anti-thrombin agent recombinant hirudin (rHir) through a bifunctional linking molecule to accessible amine functional groups, which were previously immobilized through the covalent binding of an organosilane compound to an oxidized Ti surface. Also, an alternative example is provided which illustrates the covalent juncture of Vascular Endothelial Growth Factor (“VEGF”) as part of a generated bioactive surface.

The objectives proposed and accomplished experimentally were:

Optimization of epoxysilane (Ep) binding to Ti plates (Ti-Ep);

Covalent linkage and optimization of the hydrophilic, multi-amine functional compound polyethyleneimine (PEI) to TiEp segments (Ti-Ep-PEI);

Characterization of the chemical properties of the Ti-Ep-PEI surface;

Covalent linkage of ¹²⁵I-rHir to the Ti-Ep-PEI surface;

Determination of the in-vitro anti-thrombin properties of surface bound rHir (i.e., thrombin inhibition and ¹²⁵]-rHir stability post-thrombin exposure); and

Covalent linkage of VEGF to the Ti-Ep-PEI surface.

These results demonstrated that a specific protein such as rHir or VEGF can be covalently attached to a relatively “inert” Ti surface and will retain its characteristic biological activity after attachment.

Step 1: Oxidizing at Least One Solid Surface Comprised of Titanium to Yield a Titanium Oxide Surface Layer.

A sheet of 90/6/4 Ti/Al/V alloy (11 inch×16 inch) was purchased from Titanium & Alloys Corporation (Warren, Mich.). The Ti was then thoroughly cleaned using a step-wise procedure. The Ti sheet was first cut into 5 cm×5 cm pieces. These pieces were first washed in absolute alcohol to remove stamp markings and any residual processing oils. These pieces were air-dried for 10 minutes, followed by placement into 12N hydrochloric acid (reagent grade). This acid bath was sonicated for 45 minutes, resulting in the Ti pieces changing from silver to a bluish gray color. The pieces were then washed twice in distilled water with sonication for 15 minutes. The wash bath was changed between washings. The cleaned etched pieces were then dried in an air-circulating oven at 160° C. for at least 1 hour.

Preferably, the dried plates were then immediately used and surface coated. Alternatively, plates have been stored up to one week at 160° C., in order to prevent the adsorption of atmospheric moisture, and have then been successfully coated. This cleaning procedure was employed for all Ti sheets utilized prior to silanization and amination.

Step 2: Reacting the Titanium Oxide Surface Layer with at Least One Organosilane Coupling Agent to Yield a Surface Siloxane Linkage.

### Experiment A: Optimization of Expoxysilane (Ep) Binding to Titanium (Ti) Plates to Form Ti-Ep Anchor Sites

Preparation of Epoxysilanol Solution:

The epoxysilanol solution was prepared using a binary solvent system composed of equal volumes of absolute alcohol and anhydrous isobutanol. Glycidyloxypropyltrimethoxysilane (2 g) was dispersed into 97.5 g of the solvent system using sonication. Distilled water (1 g) was then added and sonicated for 15 minutes to homogeneously distribute the water. This solution (Ep) was allowed to stand 24 hours prior to use. Solutions made in this manner have shown a shelf stability of greater than six months without turning cloudy or forming precipitate.

Silanization of Ti Plates:

The cleaned Ti pieces were preheated to the coating temperature of 160° C. in order to remove potential excess moisture. Individual pieces were removed, cooled and immediately coated with the Ep solution using a syringe. The coating technique involved holding the piece at one end using forceps while the coating was applied via syringe. The coated piece (Ti-Ep) was then held in the oven with the door open until the solvent evaporated. Each Ti piece was hung from one corner using an alligator clip attached to oven shelf. The pieces were cured at 68° C. for 1 hour before removal and post-curing. Post-curing involved incubating the Ti-Ep pieces at 160° C. for 17-24 hours. After post-curing and removal from the oven, the coated piece was immersed into boiling water and held at a rigorous boil for 15 minutes. The Ti-Ep pieces were blotted dry and wrapped in aluminum foil awaiting amination.

Results:

The Ti pieces, after alcohol cleaning and acid etching, changed from a silver color to a bluish gray color. The Ti pieces did not change color after Ep coating. Upon removal from the water bath after Ep curing, it was readily apparent that a hydrophobic coating existed on the metal surface by the rapid run off of water and the spherical beading of any remaining water droplets. Grossly, the Ti pieces had a coating across the entire surface, with some imperfections (i.e. scratches) due to handling with the forceps. The next step was to immobilize amine groups to the Ti surface using the Ep coating as “anchor” sites.

Step 3: Combining the Surface Organic Reactive Site with a Substance having at Least One Functional Amino Group Available for Subsequent Chemical Reaction.

### Experiment B: Covalent Linkage and Optimization of the Hydrophilic, Multi-Amine Functional Compound Polyethylenimine (PEI) to Ti-Ep Segments (Ti-Ep-PEI)

Procedures:

The pendant glycidol group was reacted with an abundance of an 800 molecular weight polyethylenimine (PEI) in order to promote end-capping with multiple, terminal amino groups. A 10% PEI solution was prepared in absolute ethanol and mixed through sonication. This PEI solution (100 ml) was placed into a 1000 ml beaker containing one 5 cm×5 cm Ti-Ep piece as previously described. This PEI-Ti-Ep reaction was briefly shaken, covered with aluminum foil and placed into a 68° C. oven. The Ti-Ep/PEI reaction was held at this temperature for 2 hours (Ti-Ep-PEI). The Ti-Ep-PEI pieces were then removed and washed twice with distilled water, changing the rinse solution each time. The rinsed Ti-Ep-PEI pieces were then placed into boiling water for 10 minutes to remove any non-specifically absorbed PEI, thereby leaving only covalently bound chains. The amine terminated Ti-Ep-PEI pieces were then quantified for amine content using two separate methods. Amine content was first grossly visualized using a textile dye (orcoacid phloxine or acid red 1). Once the amine groups were present, the amine content/weight segment was quantified using sulfo-SDTB (Pierce, Rockford, Ill.). The second quantification method employed x-ray photoelectron spectroscopy (XPS) also known as electron spectroscopy chemical analysis (ESCA). Both these test methods are fully explained and their respective results are given subsequently herein.

Results:

Macroscopically, the binding of PEI to the Ti-Ep segments maintained the bluish gray color that was formed initially on the Ti-Ep segments. One difference between the Ti-Ep and Ti-Ep-PEI surfaces was water retention by the surfaces. The Ti-Ep surfaces were extremely hydrophobic, with water rapidly beading off. In contrast, water association with the Ti-Ep-PEI segments persisted for a longer period of time indicating that hydrophilic properties were established by PEI binding. Thus, it appeared that PEI binding established a degree of hydrophilicity on the Ti surface. The next point was to confirm if amine groups were present.

### Experiment C: Characterization of the Chemical Properties of the Ti-Ep-PEI Surface

Part I: Determination of Total Amine Content

Procedures:

Acid Red 1 (AR1), an anionic dye, was employed to quantitatively and qualitatively assess total (primary and secondary) amine content in the Ti-Ep-PEI segments. Briefly, a 500 ml stock solution of AR1 (0.5 mg/ml, dye purity=60%) was prepared in 0.01 M MES pH 4.5 (MES). A working solution of AR1 was prepared by aliquotting 10 ml of the stock solution and bringing to a total volume of 100 ml with MES buffer (50 mg/ml). Segments (0.8 cm×1.0 cm) were cut from Ti, Ti-Ep and Ti-Ep-PEI plates (n=3/test group/treatment). Working AR1 solution (4 ml) was added to each segment and incubated for 1 hour. The segments were removed and placed into wash solution of MES buffer for one hour. Dye bath and wash solutions were read at 530 nm using MES buffer as blank. Qualitative and quantitative assessment of amine groups created on each Ti segment (nmoles/mg segment) was calculated using standard equations as previously described [Dempsey et al., ASAIO J. 44:M506 (1998); Phaneuf et al., J. Biomed. Appl. 12:100 (1997)].

Results:

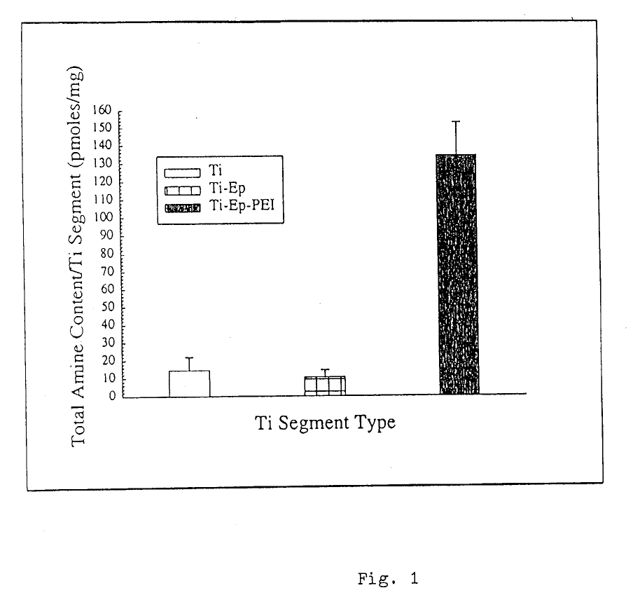

Macroscopically, Ti-Ep-PEI segments had uniform dye uptake across each segment, with some sections containing scratches due to repeated handling. The negative Ti and Ti-Ep controls had no visible dye uptake. The amount of amine groups created on the Ti-Ep-PEI segments (134±19 pmoles/mg), as determined by absorbance reduction, was 12.9 and 13.4 fold greater than Ti (15±7 pmoles/mg) and Ti-Ep (10±4 pmoles/mg) segments, confirming the observed findings. These findings are graphically shown by FIG. 1. Thus, this assay provides a rapid qualitative and quantitative determination of amine groups on the Ti-Ep-PEI surface.

Part II: Quantification of Crosslinker-Accessible Amine Content via Sulfo-SDTB

Procedures:

A stock buffer consisting of 50 nM sodium bicarbonate, pH 8.5 was prepared. Ti, Ti-Ep and Ti-Ep-PEI segments (n=3/test condition; approximate segment size=0.8 cm×1.0 cm), which were previously prepared and cut, were weighed. Sulfo-SDTB reacts with only primary amine groups, similar to the reaction mechanism of the heterobifunctional crosslinkers employed in this study. Thus, the amine content determined via this methodology was expected to be lower than the acid red study; and would provide an indication to whether or not these bifunctional linking molecules would bind to the pendant amines generated on this surface.

Sulfo-SDTB (3 mg) was weighed and dissolved in 1 ml dimethylformamide (DMF). After thorough mixing, the sulfo-SDTB solution was brought up to a total volume of 50 ml with the stock sodium bicarbonate buffer (working sulfo-SDTB solution). Stock buffer (1 ml) and 1 ml working sulfo-SDTB solution were added to each tube and reacted for 40 minutes at room temperature on an orbital shaker at 150 r.p.m.

Segments were then removed and washed twice in 5 ml of distilled water on an inversion mixer (40 r.p.m.). Immediately following the wash, 2 ml of a perchloric acid solution (51.4 ml 70% perchloric acid and 46.0 ml distilled water) was added to each segment. Segments were reacted for 15 minutes on the inversion mixer (40 r.p.m.). The reaction solution (1 ml) was then removed and absorbance at 498 nm was measured. Using the extinction coefficient for sulfo-SDTB (70,000 liters mole⁻¹ cm⁻¹) and the segment weights, amine content (pmoles)/segment weight (mg) was determined.

Results:

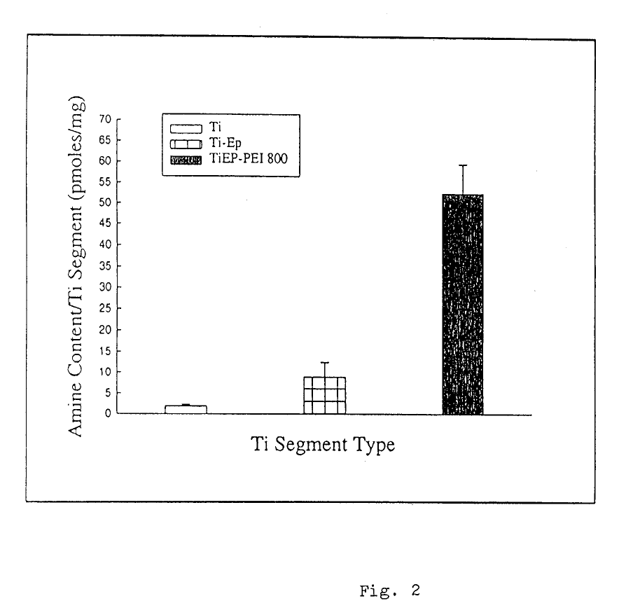

The amine content of the Ti-Ep-PEI segments (53+7 pmoles/mg) was 5.9 and 27.9 fold greater than Ti-Ep (9±4 pmoles/mg) and Ti (1.9±0.3 pmoles/mg) controls. These results are graphically illustrated by FIG. 2.

This assay provided a direct measurement of amine sites that would be accessible to heterobifunctional linking molecules. Additionally, these results confirmed the acid red studies that demonstrated that amine groups have been created on the Ti-Ep surfaces.

Part III: Surface Characterization

ESCA Analysis:

Ti and Ti-Ep-PEI samples were submitted to Analytical Answers, Inc. (Woburn, Mass.) for ESCA analysis. Both control and test samples were prepared from the same Ti sheet. Ti pieces (5 cm×5 cm) were cleaned using the alcohol and acid etching procedure. These pieces were dried in a convection oven at 155° C. overnight. A segment of the cleaned Ti piece (2 cm×2 cm) was then coated with the Ep solution. The piece was cured at 68° C. for 1 hour before removal and post-curing. Post-curing involved incubating the Ti-Ep piece at 160° C. for 17-24 hours. After post-curing and removal from the oven, the coated piece was immersed into boiling water and held at a rigorous boil for 15 minutes.

After cooling to room temperature, the Ti-Ep was placed in a 10% PEI solution. This PEI/Ti-Ep reaction was briefly shaken, covered with aluminum foil and placed into a 68° C. oven. The Ti-Ep/PEI reaction was held at this temperature for 2 hours (Ti-Ep-PEI). The Ti-Ep-PEI pieces were then removed and washed twice with distilled water, changing the rinse solution each time. The rinsed Ti-Ep-PEI pieces were then placed into boiling water for 10 minutes to remove any non-specifically adsorbed PEI, thereby leaving only covalently bound chains. Dried samples were placed in aluminum foil for transport to Analytical Answers. Analytical Answers sputtered all samples for 12 seconds using Argon.

Though a monolayer of silane coupling agent may be attainable, its necessity may not be justified. However, knowing the ratio of the atomic constituents found in the monolayer may help to define the test coating. The ideal monolayer of the coupling agent to an oxidized Ti surface would have the following atomic structure: Ti—Si—O₄—C₆. Ideally, it is expected that the ratio of Ti:Si:O:C atoms would be 1:1:4:6 for a total of 12 atoms. This can be stated in theoretical percentages as Ti=8.3%(1/12), Si=8.3%(1/12), 0=33.3%(4/12), and C=50%(6/12).

Results:

Table E-1 summarizes the results as reported by Analytical Answers. The data has been confined to the major constituents found in the Ti, Ti-Ep and Ti-Ep-PEI segments. The presence of carbon found in the control sample can be explained as contaminants. Also, the ratio of 0 to Ti in the Ti control was found to be 1.4:1, which is less than the 2:1 relationship expected to be found for TiO₂. This result is attributed to the presence of a porous Ti oxide-hydroxide or hydrate outer layer rich in water.

The Ep-coated Ti sample showed a rapid depletion in Ti indicating a coating much greater than the theoretical monolayer. Indeed, it borders on the limiting detection level for ESCA analysis of 50 to 60 angstroms. This is further indicated by the increase in the Si concentration that far exceeds the expected 8.3% found in a monolayer. The increases in O and C are expected with the formation of a multi-layered Ti-Ep coating; as well as the absence of N at the surface.

TABLE E1
ESCA Results for Major Constituents Found on Ti Samples

| | Ti % | Si % | O % | C % | N % |
|---|---|---|---|---|---|
| Ti (cleaned) | 37.5 | 0 | 53.82 | 2.51 | 0 |
| Ti-Ep | 0.88 | 20.24 | 27.13 | 51.74 | 0 |
| Ti-Ep-PEI | 0.39 | 17.35 | 23.2 | 54.26 | 3.21 |

It is noted that PEI is essentially a repeating unit represented by C₂N₂ and can be expected to be present at or very close to the surface of the Ti-Ep-PEI. Thus, the Ti-Ep-PEI surface shows a further decrease in the presence of Ti, although at a decreasing rate. Similarly, the amount of Si and O, which are not present in the PEI polymer, are expected to decrease. Also, with C and N being the only two constituents of PEI, one would expect an increase in their concentrations—as the data shows.

The sole troubling aspect is the presence of N at only a 3% level. However, in support of these results, others have shown aminosilanes existing as a monolayer attain N surface concentrations of 1 to 3% [Xiao et al., J. Mater. Sci. Med. 8:869 (1997)]. This indicates that the PEI is limited to a few angstroms of penetration into the Ep coating and perhaps exists as a monolayer. Therefore, this study in conjunction with the acid red and sulfo-SDTB results; and confirmed the presence of amine groups on the Ti-Ep-PEI surface. The next point was to show that ¹²⁵]-rHir could be covalently attached to the amine-functionalized surface.

Step 4: Covalently Joining at Least One Bifunctional Linking Molecule to the Pendant Amino Groups Immobilized on the Material Surface.

Procedures

Ti-Ep-PEI segments (0.8 cm×1.0 cm; n=24) were prepared at BMS, weighed and grouped into 2 sets (n=4 individual experiments). The stock sodium bicarbonate buffer solution, described in the sulfo-SDTB procedure, was utilized. A 20 mg/ml solution of Traut's reagent—the heterobifunctional linking molecule (B)—was prepared in the bicarbonate buffer and 2 ml was added to one set of Ti-Ep-PEI segments. To the other set, 2 ml of bicarbonate buffer alone was added to each segment. Each of these sets of segments were then reacted for 1 hour at room temperature on the inversion mixer. Once the Traut's reaction was complete, all the segments were washed twice on the inversion mixer with 5 ml bicarbonate buffer. The set of segments containing the surface bound bifunctional linking molecule (B) were then ready for subsequent reaction with and juncture to the biologically active protein of choice.

Step 5: Covalently Attaching a Recognized form of Hirudin Protein to the Prepared Surface Linkage.

### Experiment D: Covalent Linkage of ¹²⁵I-rHir to the Ti-Ep-PEI-B Surface

Part I: Protein Attachment

Procedure

Within 20 minutes after completing the covalent juncture of the bifunctional linking molecule (B) to the Ti-Ep-PEI surface as described above, a 4.68 mM ¹²⁵I-rHir solution (31% ¹²⁵I-rHir) was prepared. Sulfo-SMCC (10 mg/ml; 325.6 μl) was added to the ¹²⁵I-rHir solution and reacted for 20 minutes at 37° C. in a water bath. The ¹²⁵I-rHir-SMCC intermediate was then purified via gel filtration (PD-10 fast desalting column). Peak fractions were pooled and the ¹²⁵I-rHir-SMCC solution was diluted to a final concentration of 71.8 μM.

The ¹²⁵I-rHir-SMCC solution (2 ml) was then added to each tube and reacted for 3 hours at room temperature on an orbital shaker (150 r.p.m.). After incubation, segments were removed and washed twice in 2 ml 0.01M sodium phosphate, 0.5M NaCl, 0.05% Tween 20, pH 7.4 buffer for 15 minutes on an inversion mixer, followed by a single wash for 5 minutes with sonication. Segments were then gamma counted. Using protein concentration determined via Lowry assay and gamma counts of a set ¹²⁵I-Hir volume (i.e., specific activity), the amount of ¹²⁵I-rHir (ng)/Ti-Ep-PEI-B segment (mg) was determined.

Results:

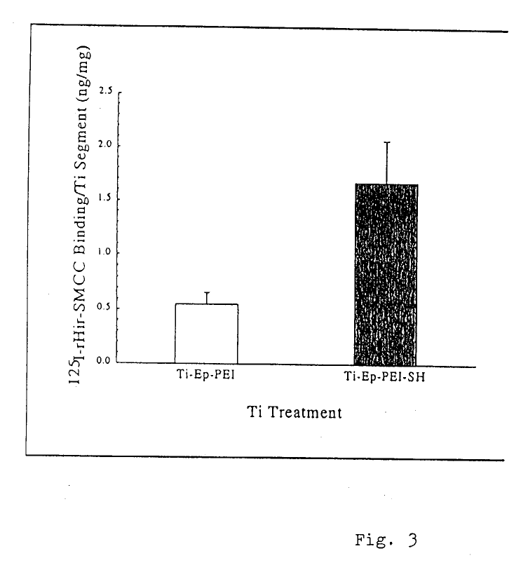

Incubation of the Ti-Ep-PEI-B segments prepared with Traut's reagent resulted in a 3-fold greater ¹²⁵I-rHir binding (1.67±0.39 ng ¹²⁵I-rHir/mg Ti) as compared to Ti-Ep-PEI segments prepared with only bicarbonate buffer. These results are graphically illustrated by FIG. 3. A normalization of the ¹²⁵I-rHir binding data across deviations in the size of the various segments was attained by incorporating the weight of the Ti segments when determining total ¹²⁵I-rHir binding a normalization of the ¹²⁵I-rHir binding data across deviations in the size of the various segments.

Interestingly, substituting sulfo-SMCC for Traut's reagent and vice-versa did not result in significant ¹²⁵I-rHir binding to the Ti-Ep-PEI-B surfaces (data not shown), potentially due to the affinity of unbound sulfo-SMCC toward the surface coating. Thus, incubation of Traut's reagent with the pre-existing Ti-Ep-PEI complex to form a Ti-Ep-PEI-B surface, which was then followed by subsequent reaction with ¹²⁵I-rHir-SMCC was the preferred and optimal procedure that resulted in a significant uptake and binding of the rHir protein. The next step was to determine if the covalently bound ¹²⁵I-rHir maintained anti-thrombin activity.

Part II: Biological Activity of the Covalently Bound Protein

Procedures

Thrombin inhibition by the Ti-Ep-PEI+¹²⁵I-rHir-SH and the Ti-Ep-PEI-B-SMCC-¹²⁵I-rHir segments (n=3/test condition) was then determined using protocols established in our previous studies [Phaneuf et al., Biomaterials 18:755 (1997)].

Briefly, each segment that was prepared was then gamma counted and placed into a 12 mm×75 mm borosilicate test tube. A stock solution consisting of 20 NIHU human α-thrombin/ml Tris buffer (0.01M Tris, 0.1M NaCl, 0.1% BSA, pH 7.4) was then made. From this stock solution, 0.5, 1.0, 2.0 and 4.0 NIHU of thrombin was added to each respective tube along with Tris buffer, bringing the total volume in the test tube to 1 ml. Positive controls (0.5, 1.0, 2.0 and 4.0 NIHU thrombin) were prepared in a similar fashion to the control and test segments. However, no Ti-Ep-PEI was added into the tube.

All reactions were then allowed to proceed for 1 hour in a 37° C. air incubator with orbital shaker (100 r.p.m.). This reaction time was selected in order to potentially provide the optimal interaction between thrombin and surface bound rHir. The solution was then removed from each tube, placed into 1 cm cuvettes, and equilibrated for 5 minutes at 37° C. in a Beckman spectrophotometer containing a thermocirculator that regulated chamber temperature. The 2.0 and 4.0 NIHU solutions are diluted to 1.0 NIHU in order to accurately measure thrombin activity.

Thrombin activity was then measured upon addition of 1 ml of 100 μM S-2238 by monitoring the change in absorbance per minute at 15-second intervals for 3 minutes at 410 nm. Thrombin inhibition was determined by the reduction in the change in absorbance per minute as compared to thrombin standards. Lastly, after incubation with thrombin, segments were washed for 10 minutes in 2 ml of the PBS/detergent buffer and gamma counted in order to determine ¹²⁵I-rHir stability on the Ti-Ep-PEI+¹²⁵I-rHir-SH and the Ti-Ep-PEI-B-SMCC-¹²⁵I-rHir surfaces.

Results:

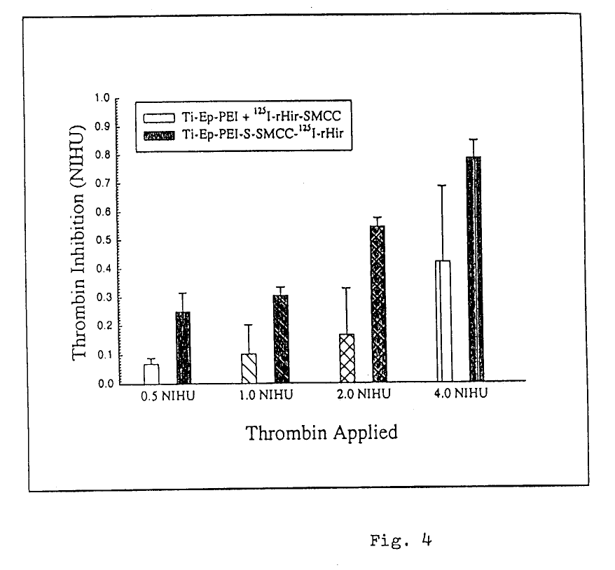

Segments with both non-specific and covalently bound ¹²⁵I-rHir inhibited thrombin throughout the thrombin concentrations assessed. The data is graphically presented by FIG. 4. The Ti-Ep-PEI-B-SMCC-¹²⁵I-rHir segments, however, inhibited significantly greater amounts of thrombin throughout all of the concentrations as compared to segments with non-specifically bound ¹²⁵I-rHir. Maximum thrombin inhibition by non-specifically bound ¹²⁵I-rHir was 0.42NIHU. Surface bound ¹²⁵I-rHir inhibited 0.79 NIH, an amount lower than the projected surface anti-thrombin properties (3.6 ATU/segment), but 2-fold greater than controls. This difference is attributed to a lack of interaction between thrombin and the Ti surface, which could be addressed by increasing reaction time and mixing parameters.

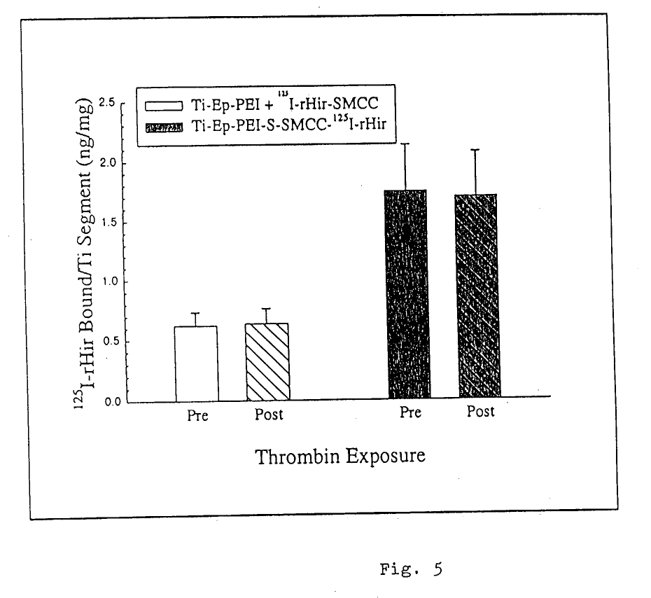

Overall, thrombin inhibition by segments with covalently linked ¹²⁵I-rHir was 1.9 to 3.6 fold greater than non-specifically bound controls. This stringent control has not been employed in other studies (which typically use unmodified Ti as the control). ¹²⁵I-rHir was not released from the surface of either non-specifically bound (0.62±0.11 ng/mg versus 0.64±0.13 ng/mg; p=0.79) or covalently bound (1.74±0.39 ng/mg versus 1.70±0.38 ng/mg; p=0.78) ¹²⁵I-rHir segments after exposure to various concentrations of thrombin. This data is illustrated graphically by FIG. 5. Therefore, these results demonstrated that ¹²⁵I-rHir covalently linked to the Ti surface maintained biologic activity and was structurally stable on the Ti surface even after interaction with thrombin.

### Experiment E: Covalently Attaching Vascular Endothelial Growth Factor (VEGF) to the Ti-Ep-PEI Surface

Procedures

Ti-Ep-PEI segments (0.8 cm×1.0 cm; n=24) were prepared, weighed and grouped into 2 sets (n=4 individual experiments). The stock sodium bicarbonate buffer solution, described in the sulfo-SDTB procedure, was utilized. A 20 mg/ml solution of Traut's reagent, the preferred heterobifunctional linking molecule (B), was prepared in the bicarbonate buffer; and 2 ml was added to one set of segments to form Ti-Ep-PEI-B surfaced segments. To other set, 2 ml of bicarbonate buffer was added to each Ti-Ep-PEI segment.

All these segments were then allowed to react for 1 hour at room temperature on the inversion mixer. Within 20 minutes of mixing completion, a 24 μM ¹²⁵I-VEGF solution (25% ¹²⁵I-VEGF) was prepared. Sulfo-SMCC (1 mg/ml; 20.8 μl) was added to the ¹²⁵I-VEGF solution and reacted for 20 minutes at 37° C. in a water bath. The ¹²⁵I-VEGF-SMCC intermediate was then purified via gel filtration (PD-10 fast desalting column). Peak fractions were pooled; and the purified ¹²⁵I-VEGF-SMCC solution was then diluted to a final concentration of 2.6 μM. After the reactions were complete, all segments were washed twice on the inversion mixer with 5 ml bicarbonate buffer.

The ¹²⁵I-VEGF-SMCC solution (1 ml) was then added to each tube and reacted for 3 hours at room temperature on an orbital shaker (150 r.p.m.). After incubation, segments were removed and washed twice in 2 ml 0.01M sodium phosphate, 0.5M NaCl, 0.05% Tween 20, pH 7.4 buffer for 15 minutes on an inversion mixer, followed by a single wash for 5 minutes with sonication. Segments were then gamma counted. Using protein concentration determined via Lowry assay and gamma counts of a set ¹²⁵I-VEGF volume (i.e., specific activity), the amount of ¹²⁵I-VEGF (ng)/Ti-Ep-PEI segment (mg) was determined.

Results:

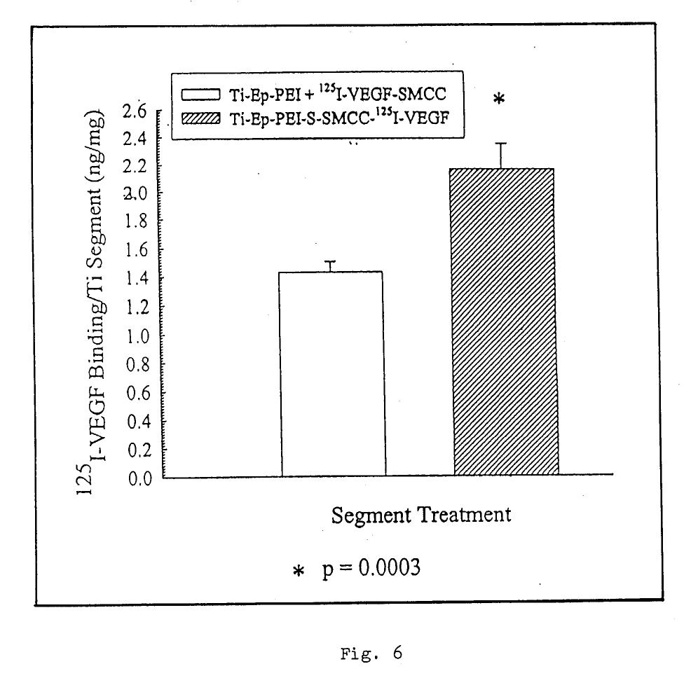

Incubation of the Ti-Ep-PEI-B segments (prepared with Traut's reagent) resulted in 52% greater ¹²⁵I-VEGF binding (2.17 ng ¹²⁵I-VEGF/mg Ti) as compared to Ti-Ep-PEI segments incubated with only bicarbonate buffer (1.43 ng ¹²⁵I-VEGF/mg Ti). These results are graphically illustrated by FIG. 6. Incorporating the weight of the Ti segments when determining total ¹²⁵I-VEGF binding permitted a normalization of the ¹²⁵I-VEGF binding data across deviations in the size of the various segments.

The present invention is not to be limited in form nor restricted in scope except by the claims appended hereto.

What we claim is:

1. A method of making a bioactive surface for a material comprised of titanium, said method comprising the steps of:
obtaining access to at least one exposed surface of a material comprised of titanium;
oxidizing said exposed surface of the material comprised of titanium with at least one oxidizing agent to yield a titanium oxide surface layer;
combining said titanium oxide surface layer with at least one organosilane coupling agent to produce a plurality of organic reactive sites disposed at the surface of the material; and
binding at least one biologically active protein to said disposed organic reactive sites to generate a bioactive surface for the material.

2. A method of making a bioactive surface for a material comprised of titanium, said method comprising the steps of:
obtaining access to at least one exposed surface of a material comprised of titanium;
oxidizing said exposed surface of the material comprised of titanium with at least one oxidizing agent to yield a titanium oxide surface layer;
combining said titanium oxide surface layer with at least one organosilane coupling agent to produce a plurality of organic reactive sites disposed at the surface of the material;
reacting said organic reactive sites disposed at the surface of the material with at least one chemically reactive composition having not less than one pendant amino group as part of its formulation and structure to yield a plurality of immobilized pendant amino groups which are functionally available for subsequent chemical reaction at the surface;
joining at least one bifunctional linking molecule to said pendant amino groups immobilized at the surface; and
binding at least one biologically active protein to said joined bifunctional linking molecule to generate an active biosurface for the material.

3. A method of making a bioactive surface for a prosthetic implant comprised of titanium, said method comprising the steps of:
obtaining access to at least one exposed surface of a material comprised of titanium in the prosthetic implant;
oxidizing said exposed surface comprised of titanium with at least one oxidizing agent to yield a titanium oxide surface layer;
combining said titanium oxide surface layer with at least one organosilane coupling agent to produce a plurality of organic reactive sites disposed at the surface; and
binding at least one recognized form of biologically active protein to said disposed organic reactive sites to generate a bioactive surface for the prosthetic implant

4. A method of making a bioactive surface for a prosthetic implant comprised of titanium, said method comprising the steps of:
obtaining access to at least one exposed surface of a material comprised of titanium in the prosthetic implant;
oxidizing said exposed surface comprised of titanium with at least one oxidizing agent to yield a titanium oxide surface layer;
combining said titanium oxide surface layer with at least one organosilane coupling agent to produce a plurality of organic reactive sites disposed at the surface;
reacting said organic reactive sites disposed at the surface with at least one chemically reactive composition having not less than one pendant amino group as part of its formulation and structure to yield a plurality of pendant amino groups which are functionally available for subsequent chemical reaction immobilized at the surface;
joining at least one bifunctional linking molecule to said pendant amino groups immobilized at the surface; and
binding at least one biologically active protein to said joined bifunctional linking molecule to generate a bioactive surface for the prosthetic implant.

5. The method of making a bioactive surface as recited in claim 1, 2, 3 or 4 wherein said oxidizing step yields a freshly generated titanium oxide surface layer.

6. The method of making a bioactive surface as recited in claim 1, 2, 3 or 4 wherein said organosilane coupling agent is epoxysilanol.

7. The method of making a bioactive surface as recited in claim 1, 2, 3 or 4 wherein said cross-linking composition having pendant amino groups is polyethyleneimine.

8. The method of making a bioactive surface as recited in claim 2 or 4 wherein said bifunctional linking molecule is selected from the group consisting of heterobifunctional and homobifunctional linking molecules.

9. The method of making a bioactive surface as recited in claim 2 or 4 wherein said bifunctional linking molecule is Traut's reagent.

10. The method of making a bioactive surface as recited in claim 1, 2, 3 or 4 wherein said biologically active protein is at least one recognized form of protein selected from the group consisting of anti-thrombin agents, thrombolytic agents, and growth promoting agents.

11. The method of making a bioactive surface as recited in claim 1, 2, 3 or 4 wherein said biologically active protein is a recognized form of Hirudin protein.

12. The method of making a bioactive surface as recited in claim 1, 2, 3 or 4 wherein said biologically active protein is at least one thrombolytic agent selected from the group consisting of streptokinase and urokinase.

13. The method of making a bioactive surface as recited in claim 1, 2, 3 or 4 wherein said biologically active protein is at least one growth promoting agent selected from the group consisting of VEGF, BMP and ECGF.

14. The method of making a bioactive surface as recited in claim 3 or 4 wherein said prosthetic implant is selected from the group consisting of surgically implantable articles of manufacture, mechanical devices, surgical implements and replacement parts.
